(19) United States
(12) Patent Application Publication
(10) Pub. No.: US 2011/0094886 A1
(43) Pub. Date: Apr. 28, 2011

(54) Automated, High Band Resolution Electrophoretic System for Digital Visualization and Method of Use

(76) Inventor: Celal Korkut Vata, Ankara (TR)

(21) Appl. No.: 12/991,132

(22) PCT Filed: May 5, 2008
(86) PCT No.: PCT/IB08/51741

§ 371 Date: Dec. 8, 2010

(51) Int. Cl. G01N 27/447; B82Y 5/00

(52) U.S. Cl. 204/603; 204/612; 204/610; 977/904; 901/2

(57) ABSTRACT

A concept of high resolution electrophoretic technique, apparatus and its application thereof. The system is a functioning entity in a radial-flat setting. Unlike the conventional rectangular gel-electrophoresis setup, wherein two electrically opposite poles are placed on opposite sites of a rectangular gel, the novel system places one of the electrical poles in the middle of the gel and the outer rims of the radial gel is exposed to the other pole. The point, central pole (positive/negative) and the radial (negative/positive) pole around the outer rim of the gel will create and maintain a gradient electric field, wherein the intensity of the electric field increases towards the central pole with an inverse proportionally to the decrease in the surface area towards the center in a radial-gradient setting. When combined with the gradient gel setup, which increases towards the center of the gel in agarose, PAGE, and in any-other media as well as gradient and non-gradient and any-other means the system has a higher resolution potential. At higher voltages moderate heating of the central electrode forms a natural heat gradient, suitable for SSCP and DGGE analyses. Additionally, radial design bestows the system better data visualization and recording potential comparable to DVD writing and reading technology.

### TECHNICAL FIELD

The invention pertains to high resolution detection and characterization of DNA, RNA, polypeptides, polynucleotides, polysaccharides, and proteins on a computerized, digitized, electrophoretic system.

### PRIOR ART

Conventional electrophoresis for the separation of biological molecules such as DNA, RNA, polypeptides, polynucleotides, polysaccharides, and proteins has been performed for many years. An electrophoresis gel assembly may include running buffers at opposite ends of the gel assembly. The gel may include a non-convective separating medium in an aqueous buffer contained in an enclosure or cassette. The opposite ends of the gel assembly are generally exposed to the running buffers. Electrodes are placed in the buffers at each end of the gel assembly with an electrical potential applied to the electrodes to impose a linear electric field, which is passed through the gel. The samples to be analyzed are typically located at one end of the gel. When the electrical charge is applied, the sample molecules migrate towards one electrode through the gel in a manner that is dependent on the potential associated with their charge and physical size. Two major types of electrophoretic separations are used: One-dimensional (“1D”) and two-dimensional (“2D”) electrophoresis. In One-Dimensional (“1D”) electrophoresis, either a tube (disc electrophoresis) or slab gel assembly is made; separations are either by one of two mechanisms: size or charge, or their combinations in terms of charge/mass ratio. In two-dimensional (“2D”) electrophoresis separation is a function of molecular weight and mainly Iso-electric point.

Electrophoresis gels were originally made from starch or cellulose. However, agarose and cross-linked polyacrylamide are widely used as the main gel materials today. Agarose is particularly useful for large double-stranded DNA (“ds-DNA”) separations. Polyacrylamide gel electrophoresis (“PAGE”) is used for higher resolution separations of somewhat smaller bio-molecules. A cross-linked polyacrylamide gel (“PAGE”) is formed by polymerizing acrylamide monomer together with a cross-linker, usually N,N′-methylene-bis-acrylamide (“Bis”). Various derivatives of these materials have been used to enhance the stability and the performance of the gel.

Laemmli in 1970 modified this assembly further to include an anionic detergent, sodium dodecyl-sulfate (“SDS”) that coats proteins to produce a uniform charge density around them. When cysteine-cysteine disulphide bonds are reduced, the proteins are separated primarily on the basis of size. These improvements have made SDS-PAGE one of the most popular and simple techniques used in biological and life sciences research.

There were two recent major advances in biomedical research, which eventually lead to the developments in proteomics and genomics technologies. i) Automated gene sequencing, and ii) gene expression profiling via microarrays. These two technologies revolutionized the fields of gene identification and disease diagnosis. Both systems are computerized and the data management is performed in silico. This feature has enabled investigators to integrate a combination of genetic information with disease diagnosis and detailed disease conditions to answer questions regarding gene-disease relationships. Further, methods, systems and devices are needed to feed the genomics and proteomics databases with recent, high quality information. Also, methods, systems and devices are required to integrate information from genomic, proteomic and traditional sources of biological information. Such information is needed for the diagnosis and prognostic prediction of diseases and other perturbations in biological systems. Conventional electrophoresis is still the most crucial technique for genomics and proteomics analysis. Especially, it is irreplaceable for confirmation of DNA-microarray and protein based expression studies.

However, the conventional electrophoretic system remains to be laborious, prone to human based errors and interpretation requires trained labor. Furthermore, gel electrophoresis is also limited by its ability to differentiate minute variations, such as post-translational modifications as well as point mutations. In conjunction with automated gene sequencing and automated gene expression profiling technologies, there is an urgent need for the automatization and computerization of gel electrophoresis.

### OBJECT OF THE INVENTION

The instant invention pertains to a method and application of a radial, computerized electrophoresis technique including a radial, slab gel structure, a central pole and a round circumferential pole around the rims of the gel as well as a computerized data mining system and associated electronic setup making use of the, DVD writing and reading analogy of the radial gel-system.

Further embodiments of the system include but are not limited to; a natural radial electric filed gradient. In a radial setting, towards the center the electric field increases in strength proportional to the decrease in the surface area in consecutive circumferential cross sections.

It is an object of this invention to make use of moderate heating of the central electrode and resulting heat gradient from center to outer rims. This is a convenient property for temperature denaturing mutation detection techniques such as SSCP and DDGE, which are applicable to point mutation detection.

It is a further object of this invention to provide an alternating pole setting, wherein switching the poles from “−” to “+” and vice versa analogues to pulse chase approach is a convenient property of the system.

It is a further object of this invention to provide a universal standard for cross comparison of experiments. One reference dimension (R) of the system enables cross comparison of experiments across different lab-platforms. Upon correcting for radius (R), experiments across from different labs can be correlated, i.e. superimposed.

Another aspect of the gel involves automated sample loading, wherein “1 well-degree rotation” of the gel facilitates the robotic arm to load samples automatically.

A further aspect of the present invention includes rotational data mining, which consists of rotation of the gel and synchronized exposure from an illuminator in concordance with a scanner to record the sample images digitally.

High sample resolution capacity of the system is a convenient feature for analytical resolution of DNA, RNA and particularly proteins.

Two 2-D applications, i) from gel to gel and ii) along the circumference from radius to radius on semi-circular orbits is a unique and a novel feature of the system with an improved 2-D resolution.

While the invention is described in connection with certain preferred embodiments, it will be understood that we do not intend to limit the invention to those embodiments. On the contrary, we intend to cover all alternatives, modifications and equivalents as may be included within the spirit and scope of the invention as defined by the appended claims.

We also do not limit the invention by automization, only. Applications of automization can be performed manually if preferred. Terms “pre-cast”, “manual”, “automated” do not intend to limit the application and can be used differentially with respect to preferred application of the system.

It will be readily apparent to those skilled in the art that still further changes and modifications in the actual concepts described herein can readily be made without departing from the spirit and scope of the invention as defined by the following claims.

### BRIEF DESCRIPTION OF THE DRAWINGS

FIG. 1 a is a graphical representation of the radial gel concept.

FIG. 1 b is a visual representation of the gradient electric field.

FIG. 2 is a schematic view of the invention with respect to conventional system.

FIG. 3 is an advantageous depiction of the surface area of radial and linear systems.

FIG. 4 is a diagrammatic representation of the classical gel apparatus in the radial setting.

FIG. 5 is a graphical representation of the radial gel chamber.

FIG. 6 is a flowchart scheme of high resolution, radial band display technique and its applications.

FIG. 7 is a depiction of the automated loading application of the system.

FIG. 8 a is an illustration of the high resolution capacity of the radial system.

FIG. 8 b is an experimental confirmation of radial high resolution concept.

FIG. 8 c is an experimental comparison of high band resolution concept.

FIG. 9 a is a visual representation of experimental gel images (2% Agarose) of radial and linear setting in concordance with theoretical graphical illustrations.

FIG. 9 b is a visual representation of quantification of radial migration pattern with respect to linear migration pattern.

FIG. 10 is an illustration of migration pattern (Log Molecular Weight Versus Migration Distance.)

FIG. 11 is an experimental illustration of linear migration and radial migration patterns on 10% denaturing PAGE.

FIG. 12 a is a particularly advantageous depiction of the radial gradient gel system.

FIG. 12 b is an experimental depiction of the gradient gel effect (4%, 5%, and 6% agarose).

FIG. 13 a is a graphical illustration of reverse running pattern.

FIG. 14 is an experimental illustration of the differential resolution with respect to radius (R).

FIG. 15 a and FIG. 16b are an experimental illustration of 10% SDS PAGE gels Stained with coomassie blue

FIG. 17 is a diagrammatic view of the invention and its application in diagnostic front.

FIG. 18 is a detailed depiction of radial symmetry and its use in the system.

FIG. 19 is graphical depiction of differential band display concept.

FIG. 20 is a schematic depiction of the radial display technology.

FIG. 21 is a graphical representation of the rotational data mining concept

FIG. 22 is a visual representation of DNA bands with respect to the radial coordinates.

FIG. 23 is a a schematic representation of barcode-based differential data-mining on radial band display setting.

FIG. 24 is a visual representation of the radial coordinates and automated digital data visualization.

FIG. 25 is a graphical depiction of the radial system with respect to electro-blotting.

FIG. 26 is a graphical description of the systems application in autoradiography.

FIG. 27 is an illustration of the radial electric field from radius to radius.

FIG. 28 is an advantageous depiction of the semi radial 2-D application of the system.

FIG. 29 a, FIG. 29 b, FIG. 29 c are experimental results of semi radial 2-D application of the system.

FIG. 30 is an alternative 2-D application of the radial system.

FIG. 31 is a graphical illustration of universal comparison across from different laboratory platforms.

FIG. 31 is an experimental illustration of universal comparison across from different laboratory platforms.

FIG. 32 is a particularly advantageous embodiment of the radial system illustrated showing heat gradient due to the heating of the central electrode

FIG. 33 is a graphical depiction of the system and its application in an automated sample locator and differential sample excision.

FIG. 34 is a graphical illustration of the sequencing application of Sanger Di-Deoxy sequencing technology.

FIG. 35 is a possible application of the system at the micro scale. Microfludic loading of the gel, gel visualization and digitation of the image is depicted here.

FIG. 36 is a diagrammatic representation of the directional running of the system.

FIG. 36 a is a depiction of the system in the forward running setting.

FIG. 36 b is an illustration of the system in the reverse running setting.

FIG. 36 c is a depiction of pulse filed application on the radial gel.

FIG. 37 a is a visual representations of conical capillary tubes making use of the radial-gradient electric field.

FIG. 37 b is a visual representations of conical radial-gradient electric field in a conical capillary.

FIG. 37 c is showing aligned individual capillaries in a radial display setting.

### DETAILED DESCRIPTION OF THE INVENTION

A concept of high resolution electrophoretic technique, apparatus and its application thereof. The system is a functioning entity in a radial-flat setting. Unlike the conventional rectangular gel-electrophoresis setup, wherein two electrically opposite poles are placed on opposite sites of a rectangular gel, the novel system places one of the electrical poles in the middle of the gel and the outer rims of the radial gel is exposed to the other pole. The point, central pole (positive/negative) and the radial (negative/positive) pole around the outer rim of the gel will create and maintain a “gradient electric field”, wherein the intensity of the electric field increases towards the central pole with an inverse proportionally to the decrease in the surface area towards the center in a radial-gradient setting. When combined with the gradient gel setup, which increases towards the center of the gel in agarose, PAGE, and in any-other media as well as gradient and non-gradient and any-other means the system has a higher resolution potential. At higher voltages moderate heating of the central electrode forms a natural heat gradient, suitable for SSCP and DGGE analyses. Additionally, radial design bestows the system better data visualization and recording potential comparable to DVD writing and reading technology.

This invention aims to fulfill the need for a high resolution, automized electrophoresis system, with an improved band resolution and visualization. Prior art aiming to improve electrophoretic resolution mainly focuses on modifications of the separation medium and buffer conditions (U.S. Pat. No. 5,219,923, U.S. Pat. No. 0,131,553 A1). However, there are no other studies utilizing radial, gradient electric field for improved electrophoretic resolution in a gradient setting. We have applied a point like central electrode and a circular electrode in a horizontal slab setting to the conventional electrophoresis system. From design point of view closest prior art (EP1217367A1) is in a cylindrical, tubular setting with an elongated central electrode and surrounding 3D electrode. Functionally, the design is aiming heat reduction and membrane to membrane transfer of biomolecules in a chromatographic manner at the industrial scale rather than high resolution analytical electrophoretic separation on a flat horizontal front making use of the “gradient nature” of the radial electric field. Prior art (EP1217367A1, U.S. Pat. No. 3,616,453, and U.S. Pat. No. 3,844,926) has used cylindrical, tubular design to increase surface area for heat exchange, mainly in industrial scale. Our invention is unique in using radial-gradient electric field for high resolution purposes, which is particularly suitable for diagnostic front. Current invention applies relatively stronger electrical force onto biomolecules as they migrate through the gel and result in differential high resolution. Furthermore, “radial band display approach” is a novel visualization concept, which can be applied to digital data mining approaches analogues to DVD writing and reading technologies. Prior art (EP1788395A1, US 20070240991) aiming to improve the gel visualization is mainly focused on gel staining techniques. There are no radial-electrophoretic designs aiming to display electrophoretic bands analogues to DVD writing and reading technology.

Unlike high throughput designs trying to eliminate heat formation we are utilizing radiating heat from the center to improve applications of temperature gradient electrophoresis and its versions such as SSCP and DDGE. As the bands migrate towards the center they are dissolved into single strands. The inverse correlation of gradient gel and the gradient electric field is the crucial point of high band resolution and such a radial visualization is unique to our system. Improved band resolution for relatively shorter samples towards the center is particularly suitable for sequencing purposes. The invention is also particularly advantageous for automatization of sample loading and gel-extraction due to its radial design. Prior art on gel atomization (U.S. Pat. No. 5,858,189, WO/1995/020155, U.S. Pat. No. 6,761,810 B2) is inefficient due to 2 linear dimensions (X and Y). Our system functions very efficiently on (R) as one dimension.

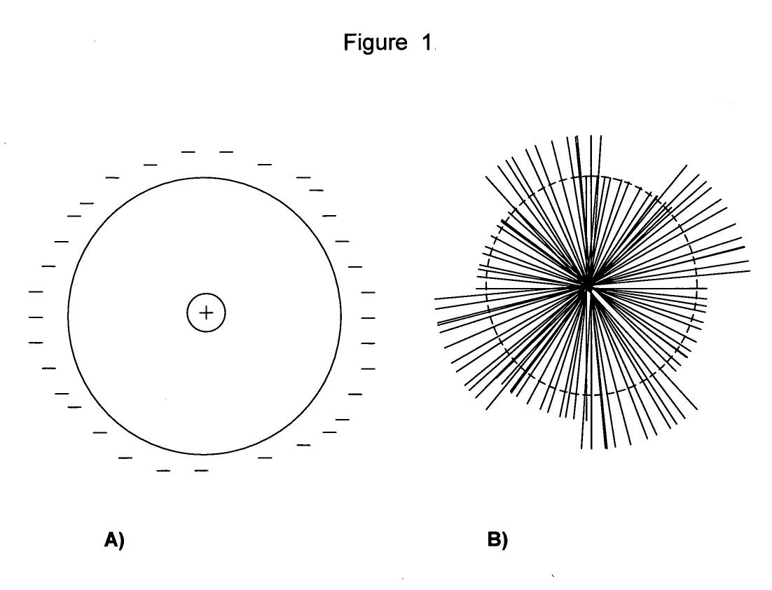

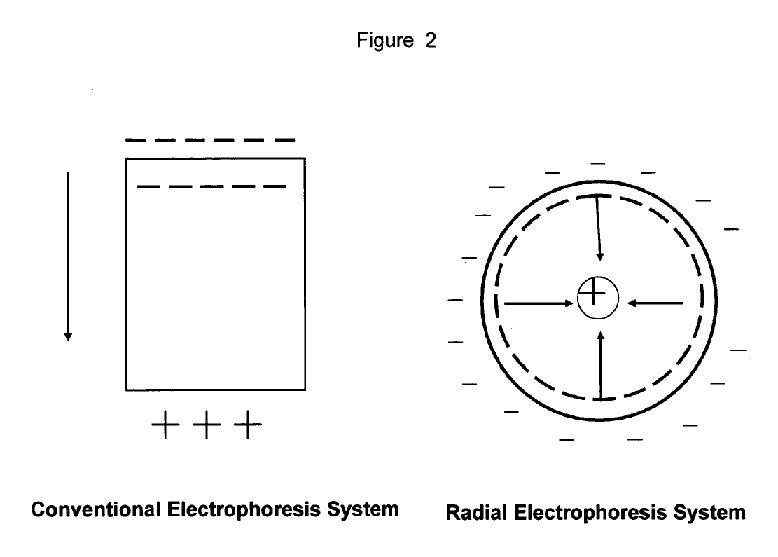

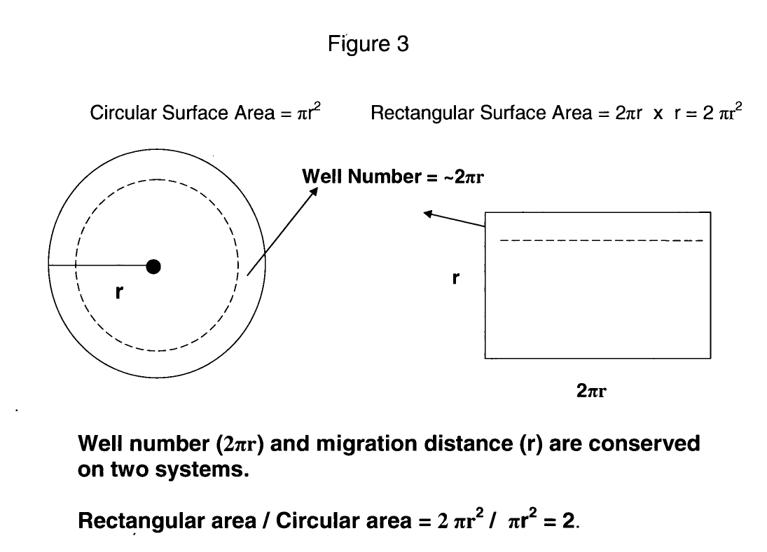

The invention pertains to an automated, high band resolution electrophoretic system for digital visualization and its method of use. The said invention is base on the radial gel concept. As a function of the radial setting, associated central electrode and the planar circular electrode at the circumference, a gradient electric field is formed. The intensity of the electric field increases towards the center with an inverse proportionality to the surface area in concentric cross sections through the system (FIG. 1 A, B). Compared to the conventional electrophoresis the invention is more compact (FIG. 2 A, B). Preserving the well length and migration distance the invention, the radial system requires half the surface area of the conventional rectangular system. Hence, the sample number is doubled in the invention, the radial design (FIG. 3). This feature is convenient for statistical studies, where high number of sample are analyzed on the same platform.

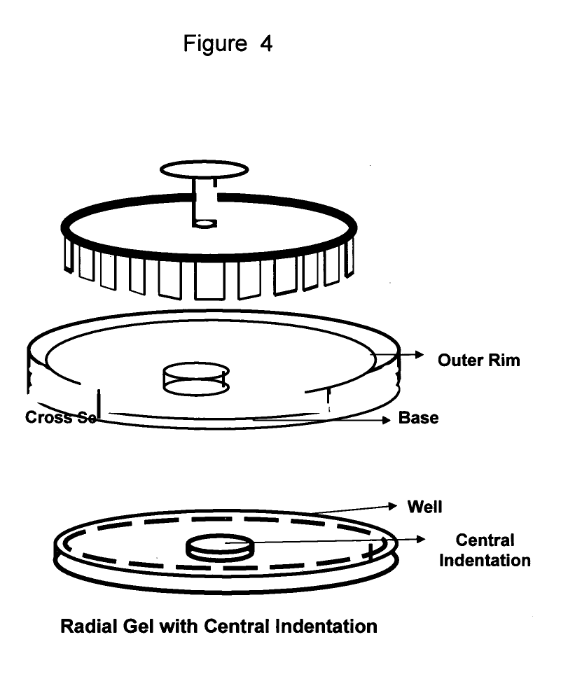

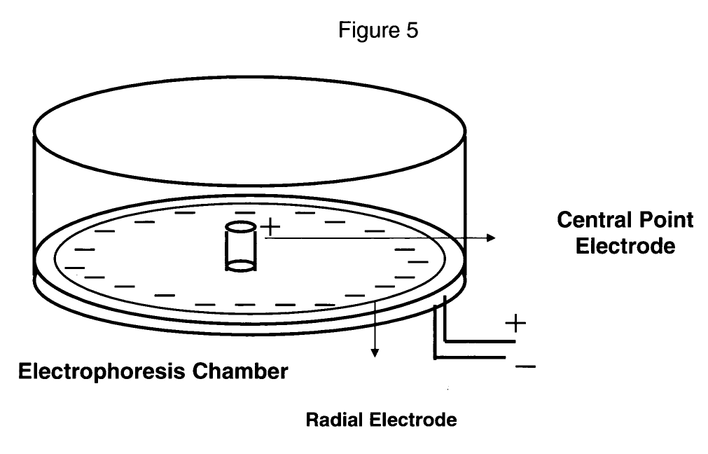

The apparatus of the invention includes all of the set up used for the conventional system. Gel casting tray, buffer chamber, combs, gels, and two electrodes. All the equipment is formulated in radial form according to the radial system. (FIG. 4, FIG. 5)

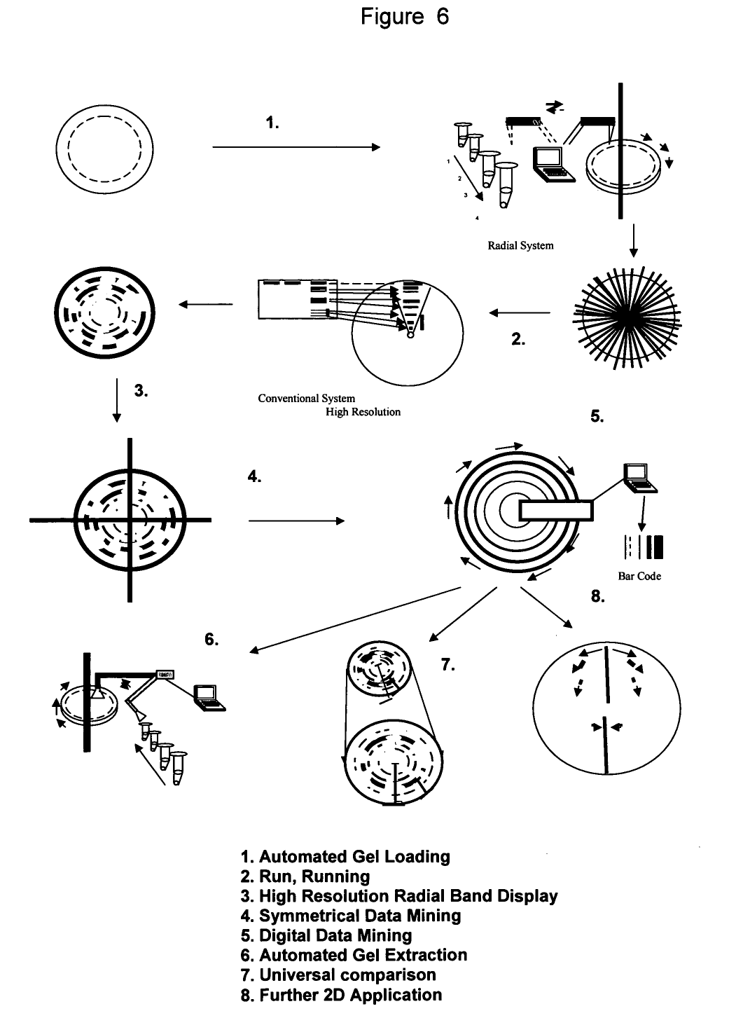

Due to the radial display of the radial system, there are broader applications, which are not functional on the conventional rectangular system. These include; 1. automated gel loading, 2. radial run, 3. high resolution radial band display, 4. symmetrical data mining on radial coordinates, 5. digital data mining, 6. automated gel extraction on radial coordinates, 7. universal gel comparison, and 8. radial 2-D analysis. (FIG. 6)

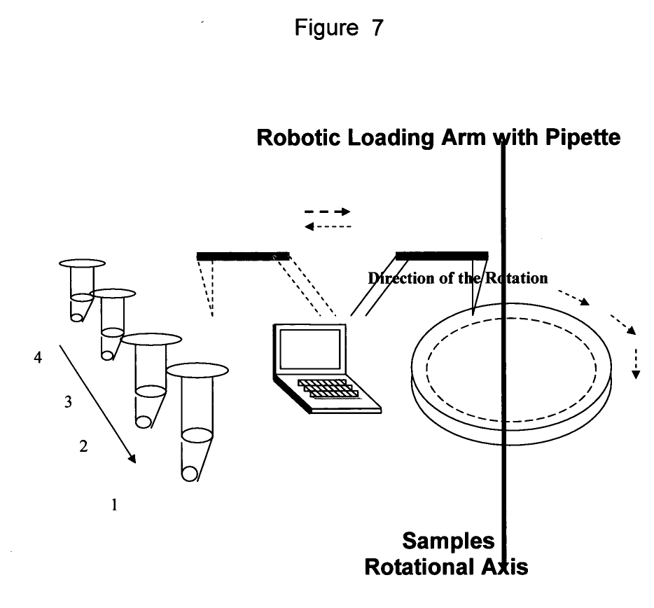

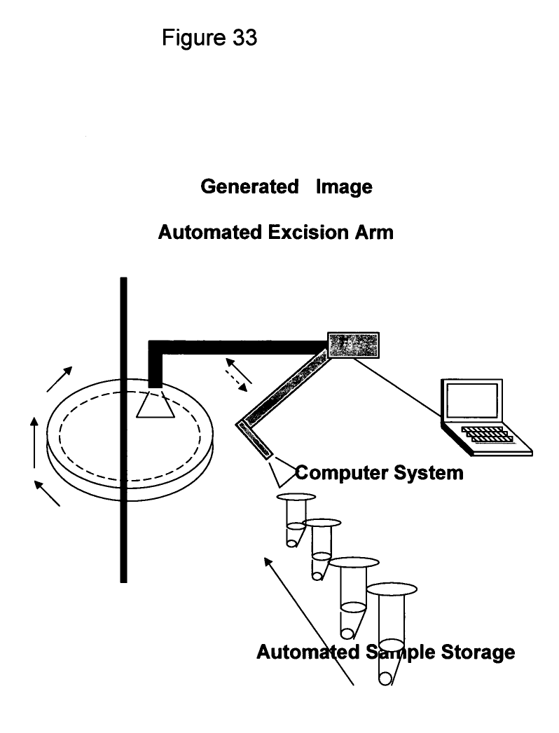

Radial rotation of the system facilitates automated gel loading, where samples can be loaded by one “Well-degree rotation” of the system (FIG. 7). Rotation of the system further facilitates automated gel loading and band extraction (FIG. 33).

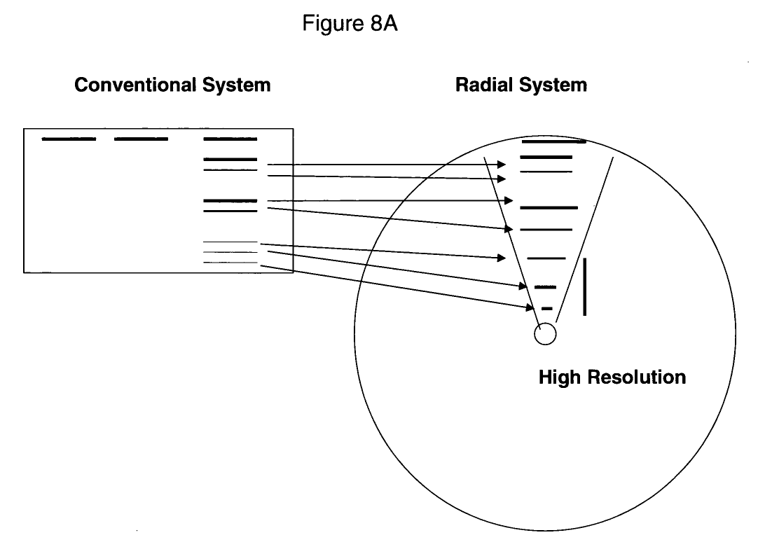

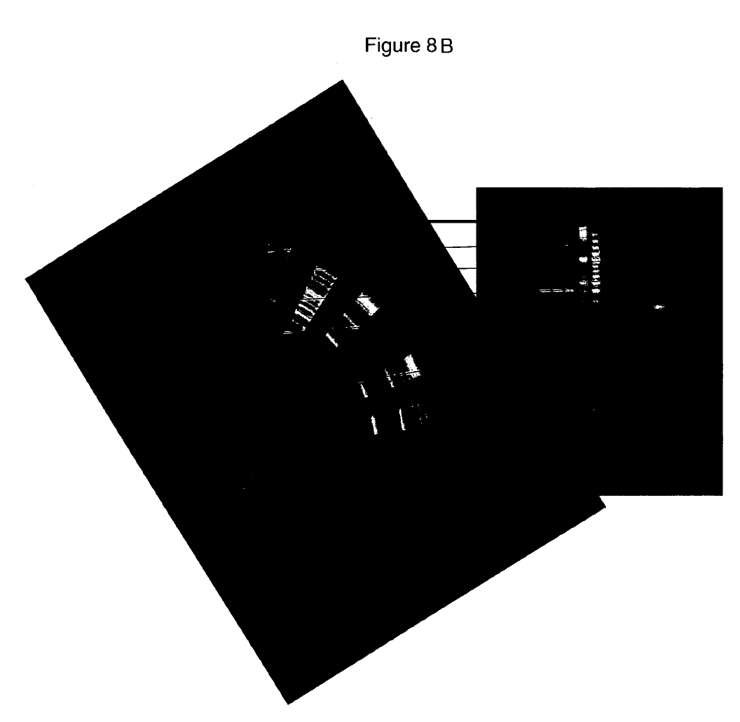

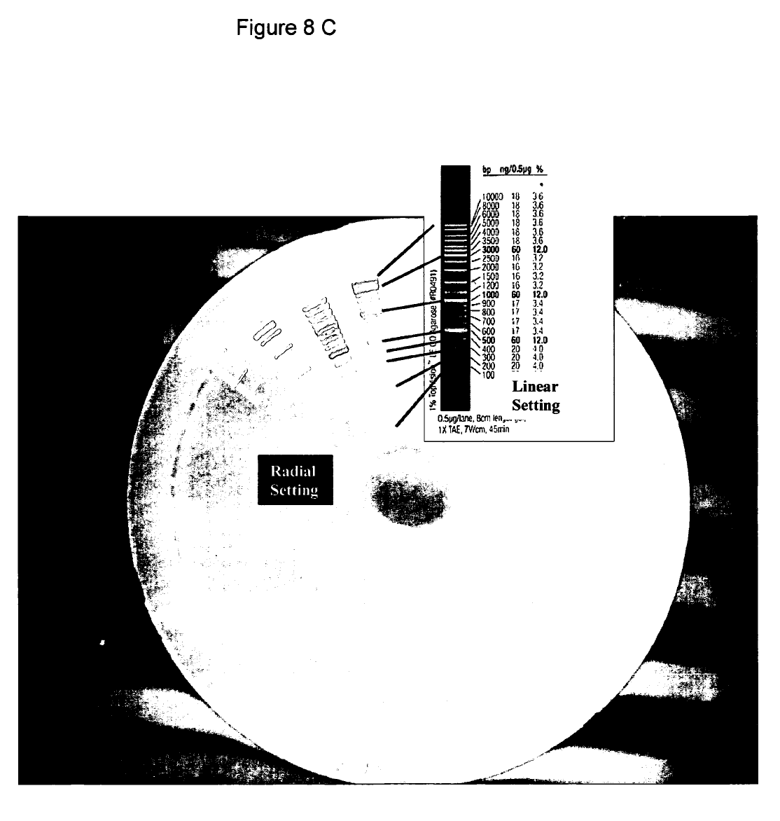

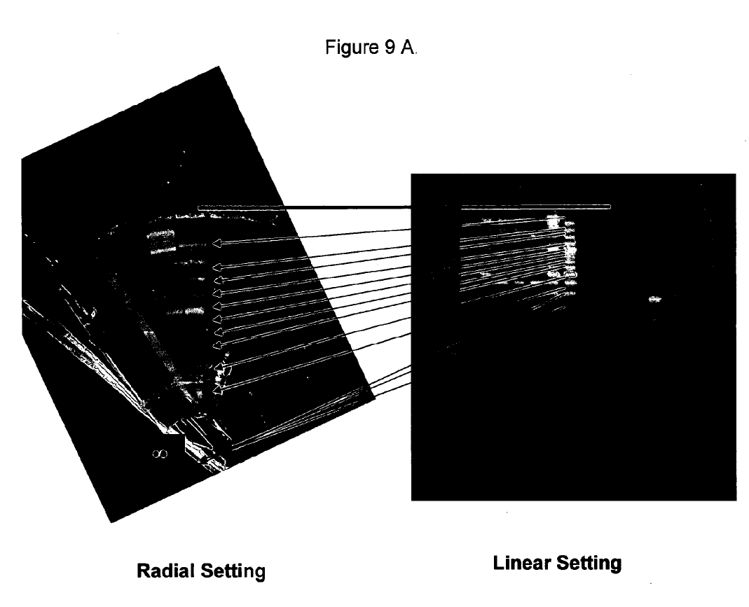

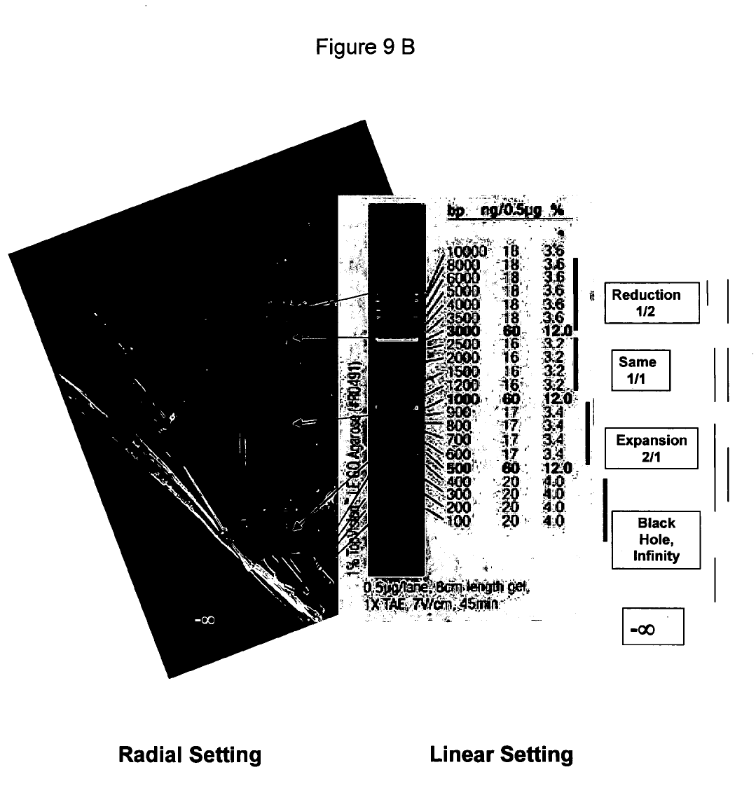

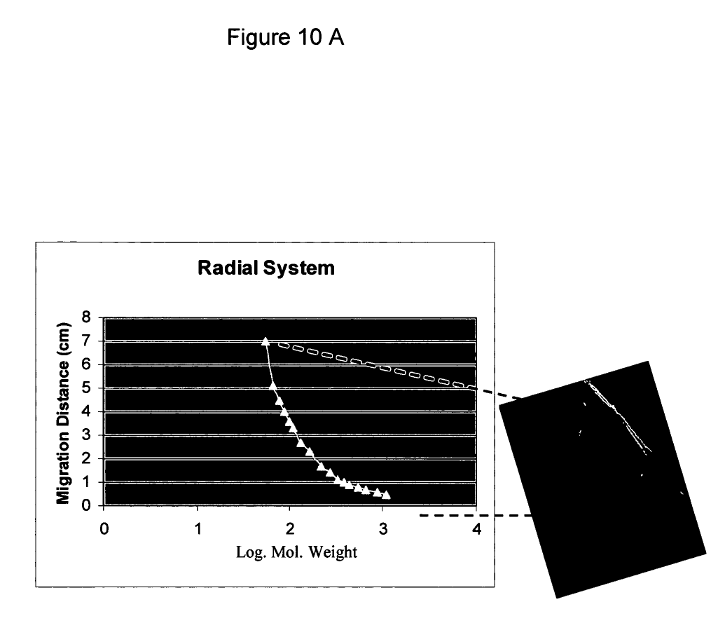

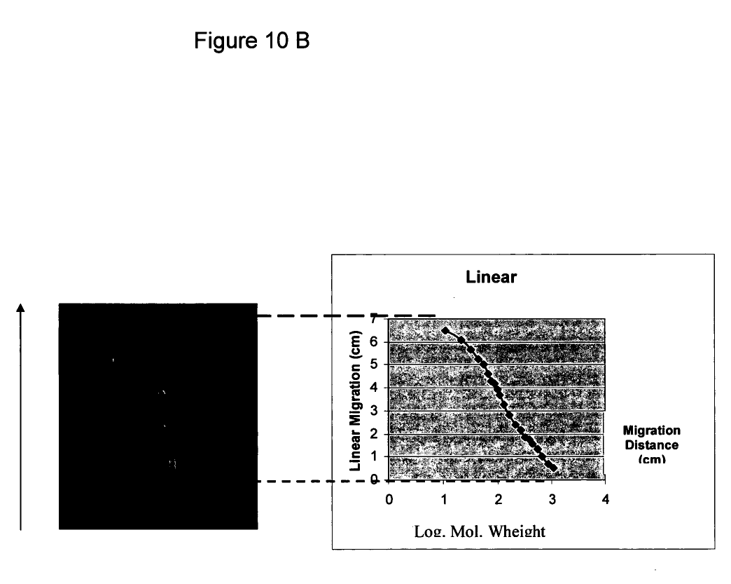

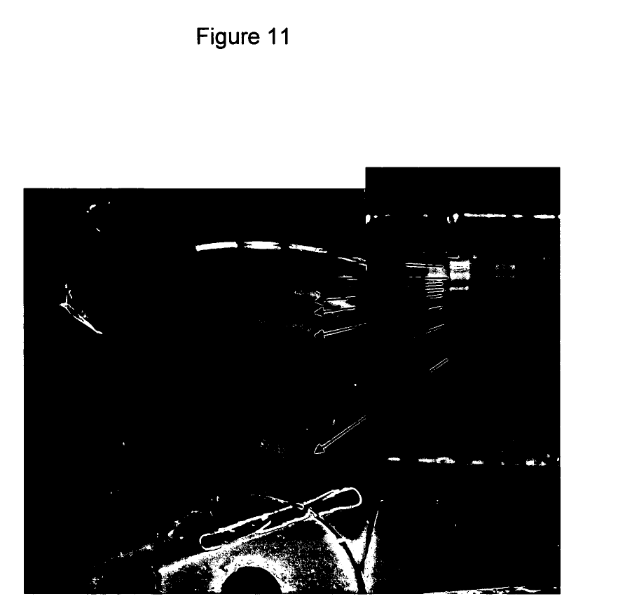

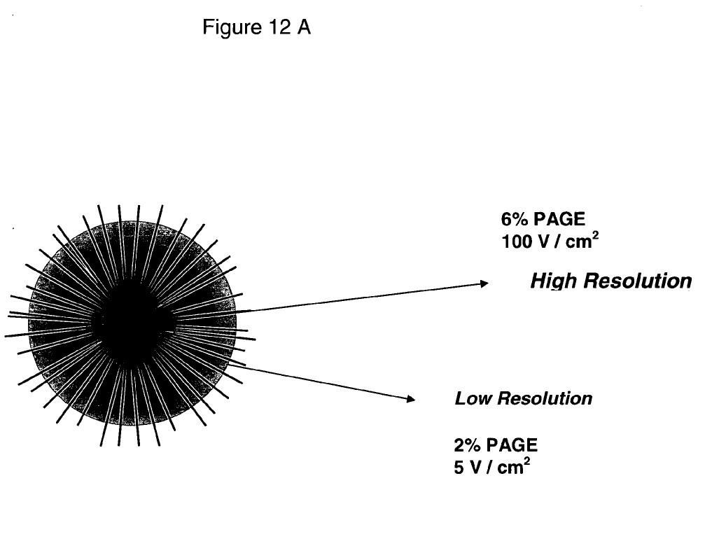

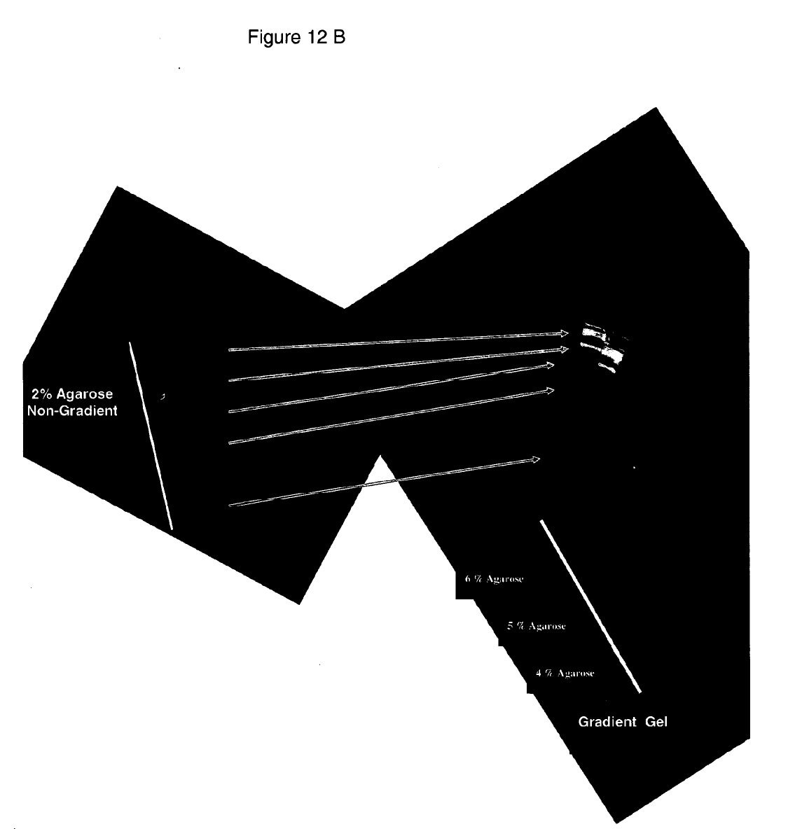

The samples are run in an accelerated fashion due to the radial electric field. As a result of the radial migration, samples of interest are resolved better towards the lower molecular weight end. This resolution is displayed in a radial setting, further dissecting bands of interest. (FIG. 8 A, B, C., FIGS. 9, 10, and 11). A gel gradient can be superimposed against electric field gradient. (FIG. 12 A, B). This feature increases the band sharpness.

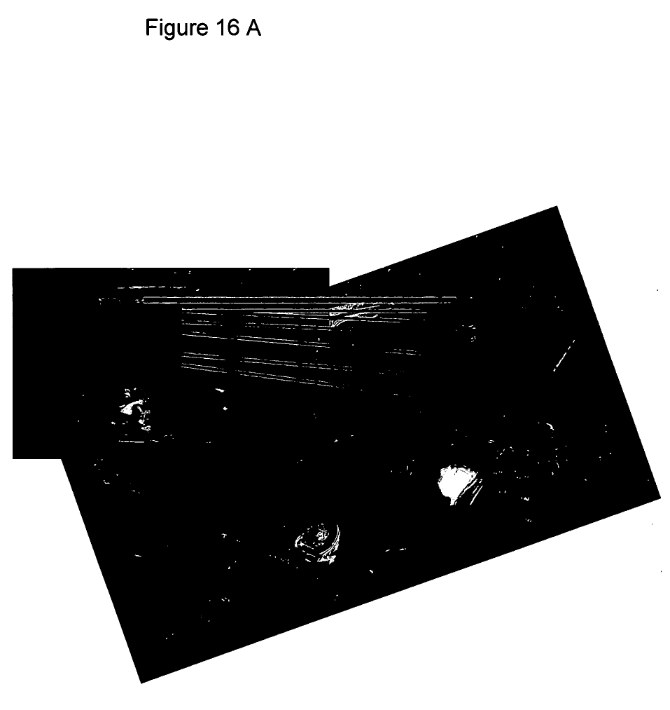

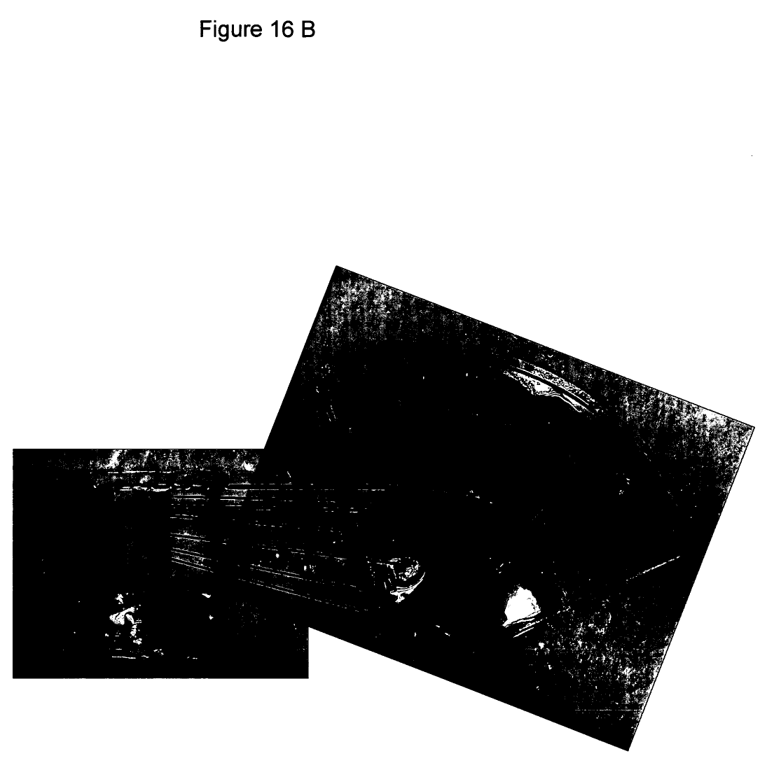

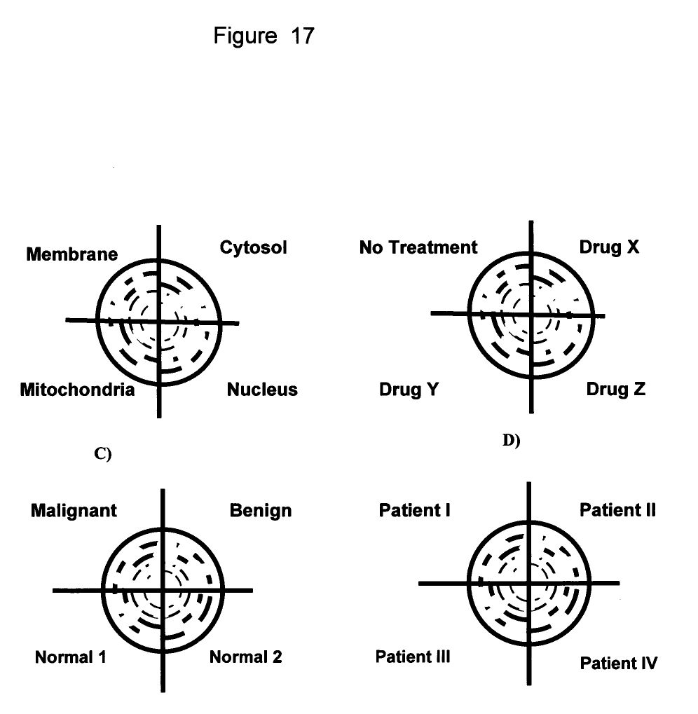

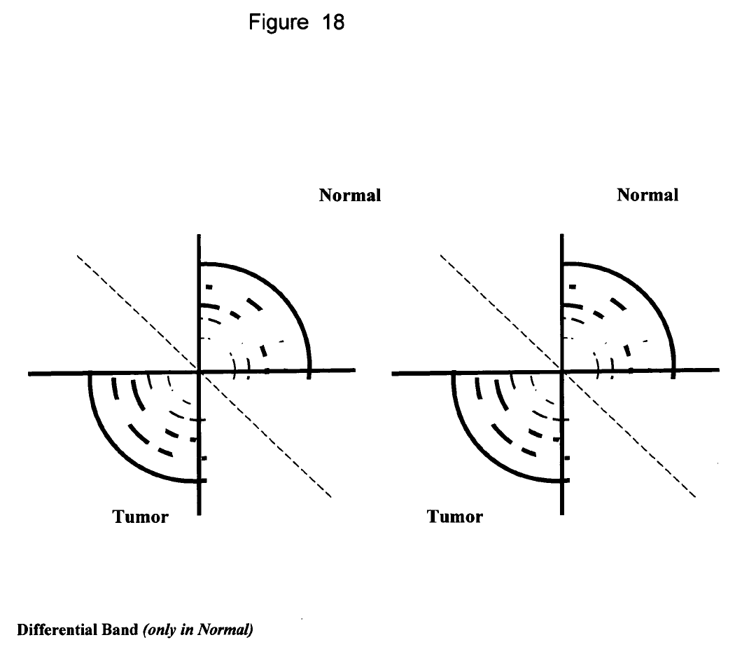

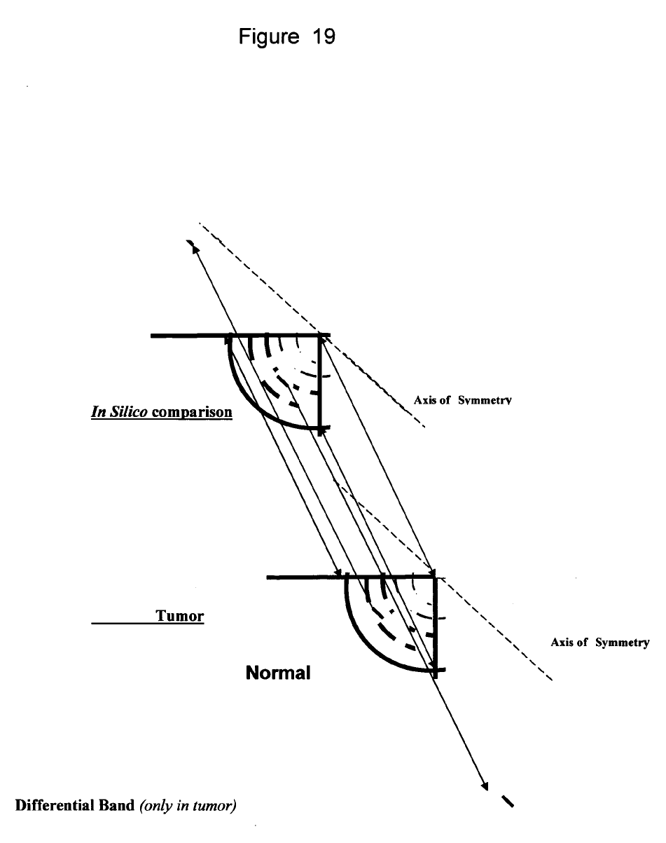

Due to the radial band display feature of the system, normal and disease samples (e.g. cancer) can be analyzed at opposite poles facilitating a superior dissection between normal and disease samples (FIG. 17, FIGS. 18 and 19).

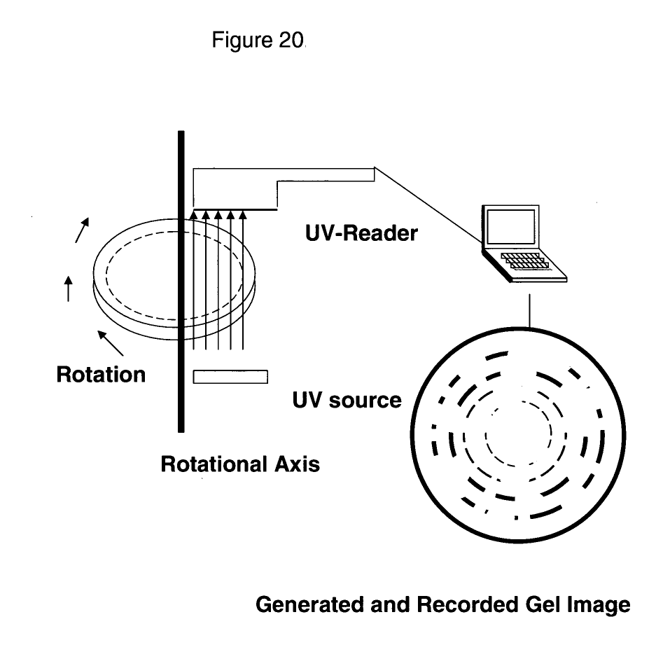

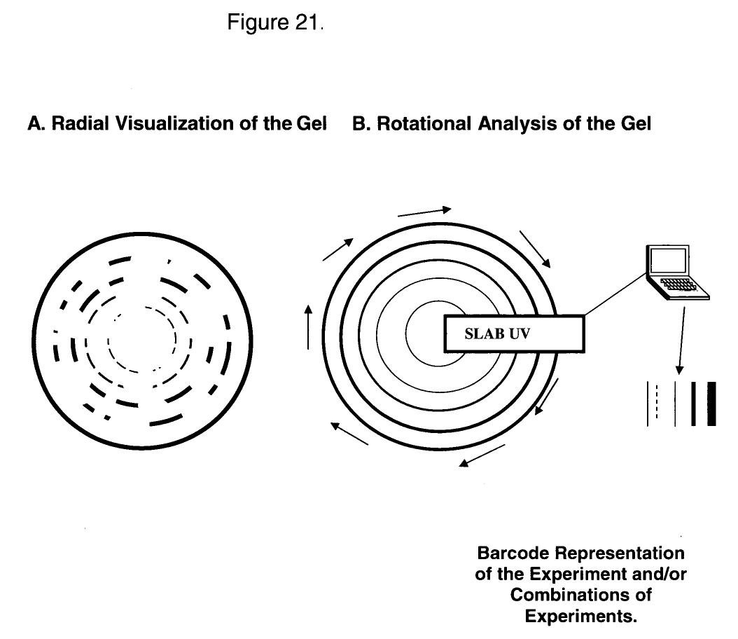

As a function of the radial display, the invention facilitates a medium, where the gel can be rotated and information is extracted analogues to the DVD technology. This feature is indicated as a barcode setting. (FIG. 20, 21)

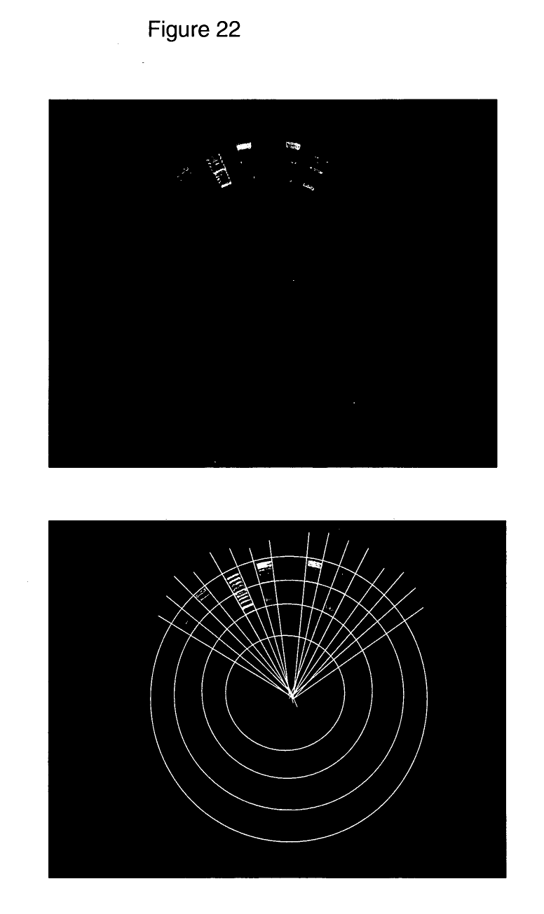

A further aspect of the radial display includes radial coordinates, where bands of interest can be located on the gel. This feature is based on radius and concentric cross sections as coordinates. Radial coordinates bestow the system a compact localization of the bands of interest. Furthermore, based on the radial coordinates an automated gel localization and gel extraction system can be applied onto the invention, the radial system. (FIG. 22).

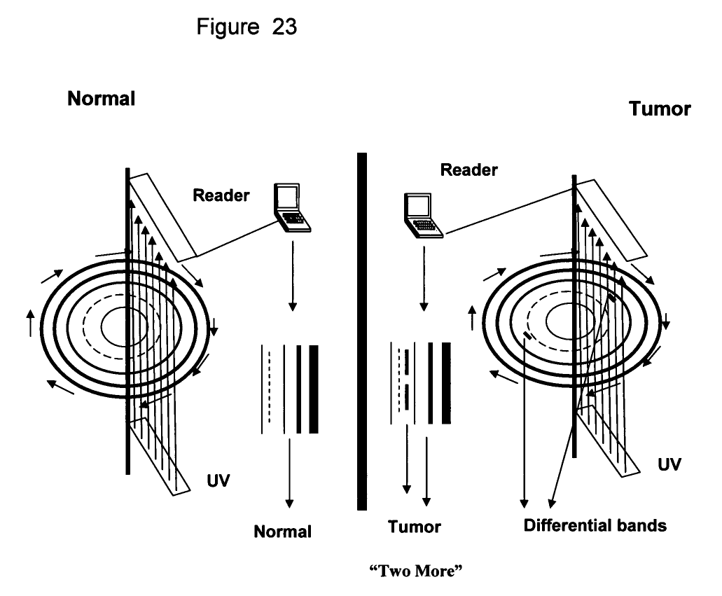

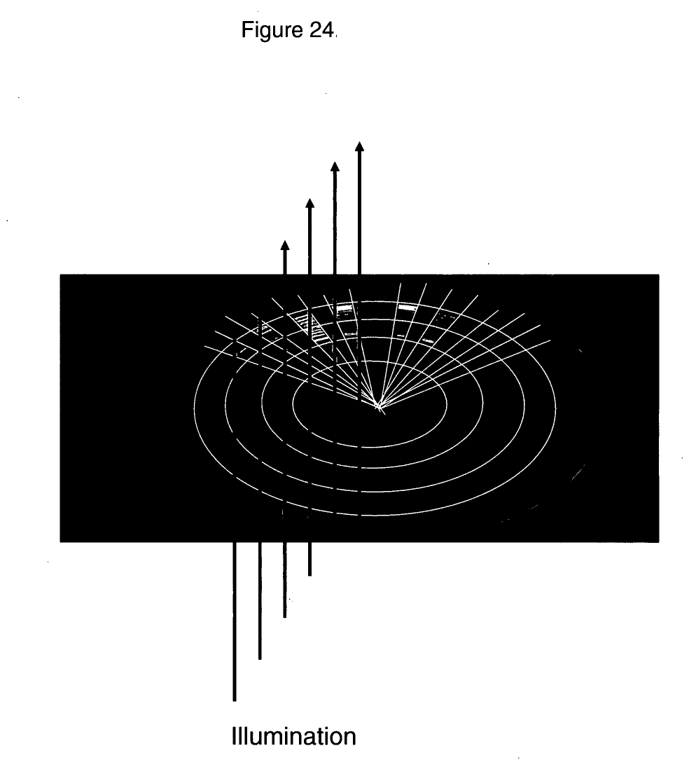

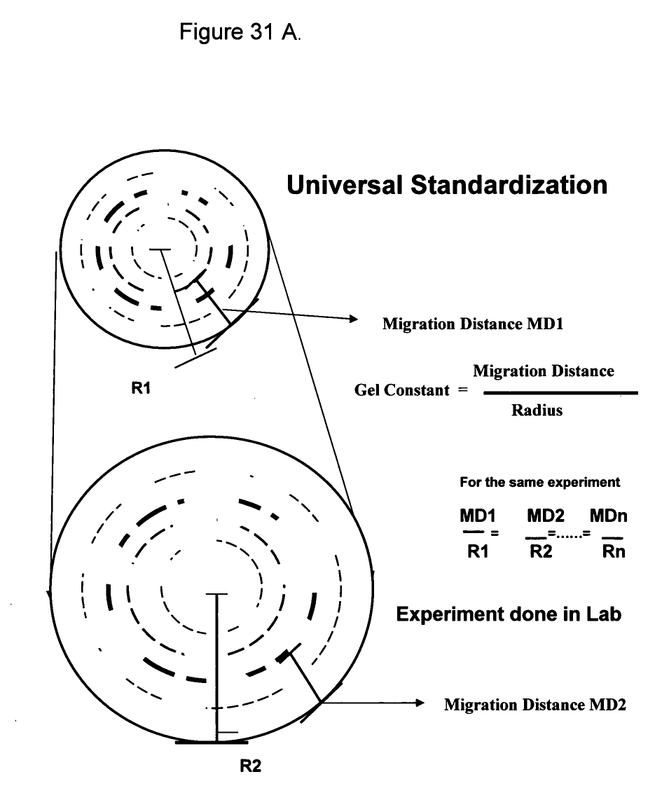

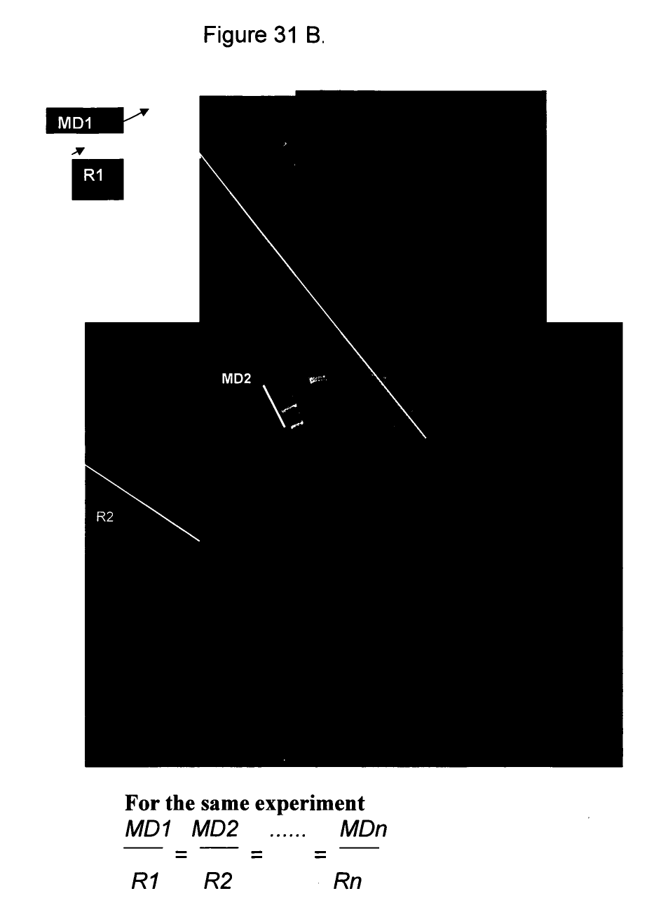

Due to the radial system and, the radius −R as the only variable coordinate, gels from across different laboratory platforms can be compared. This facilitates a universal currency, where gel images across from different laboratories can be compared. (FIG. 23, 24, FIGS. 31 A, and B.)

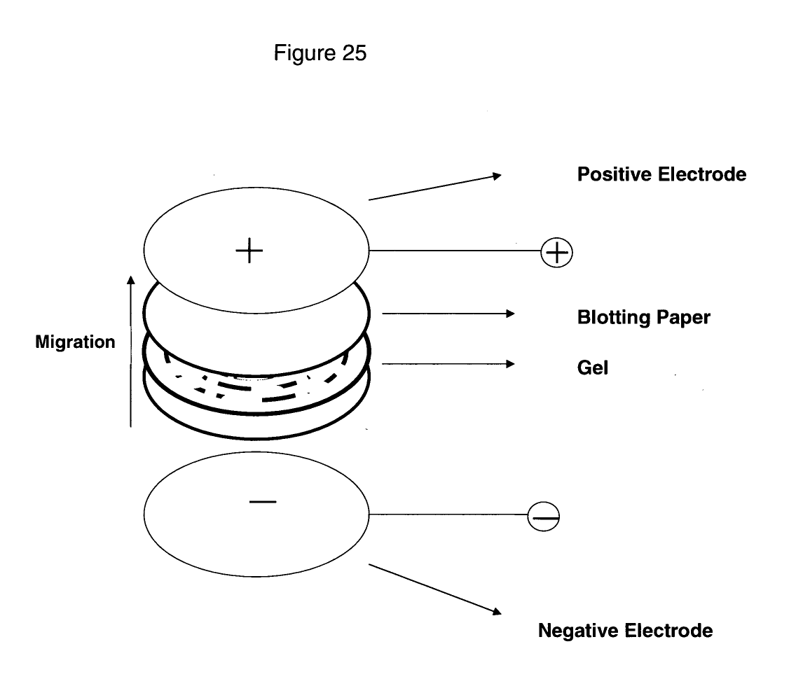

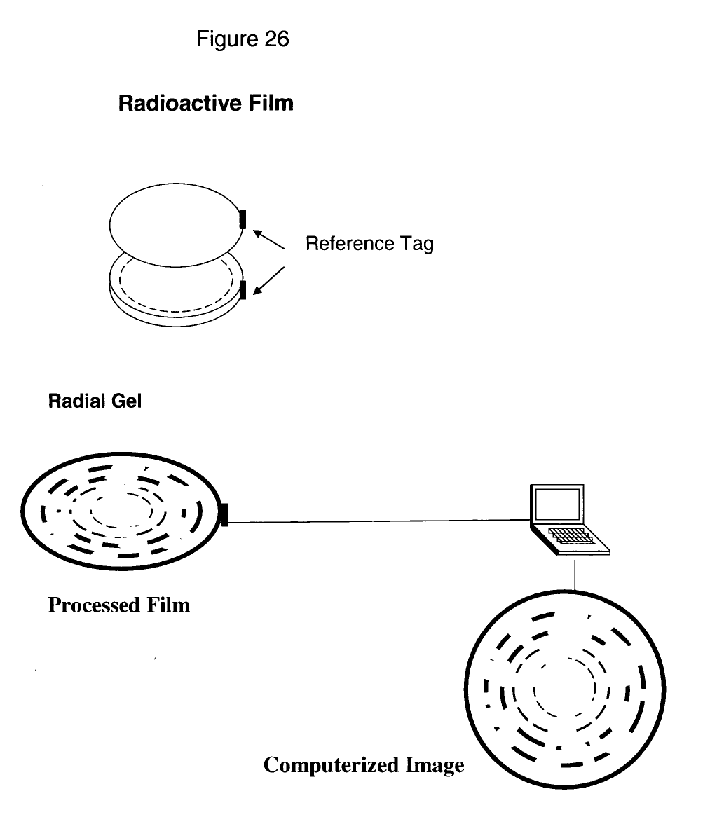

The invention, the radial system has two 2-dimensional (2D) applications. In one form radial gels can be arranged on top of each other after the first dimensional run and second dimension analysis is performed in a cylindrical setting. (FIG. 25).

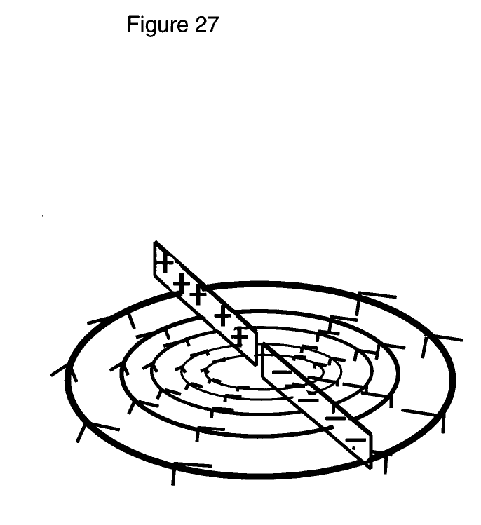

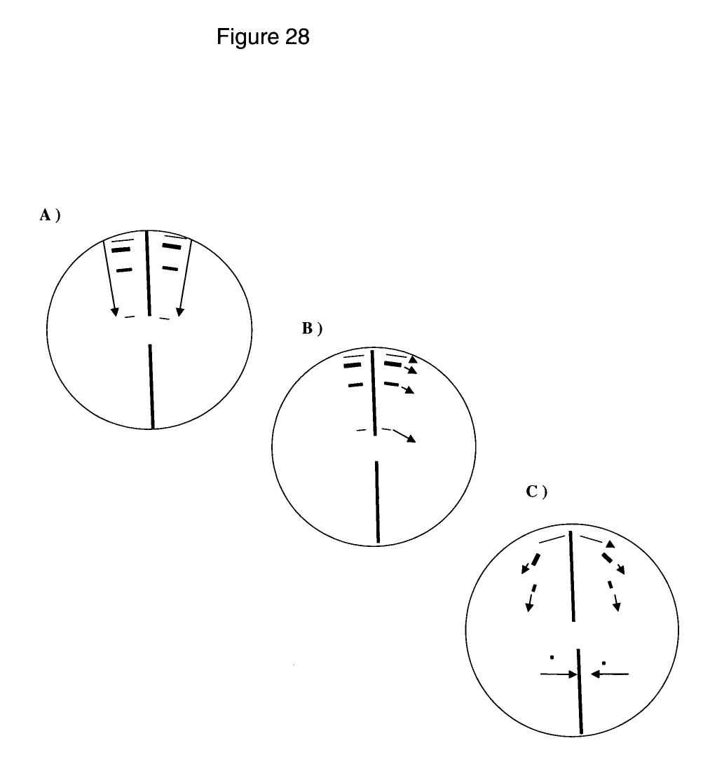

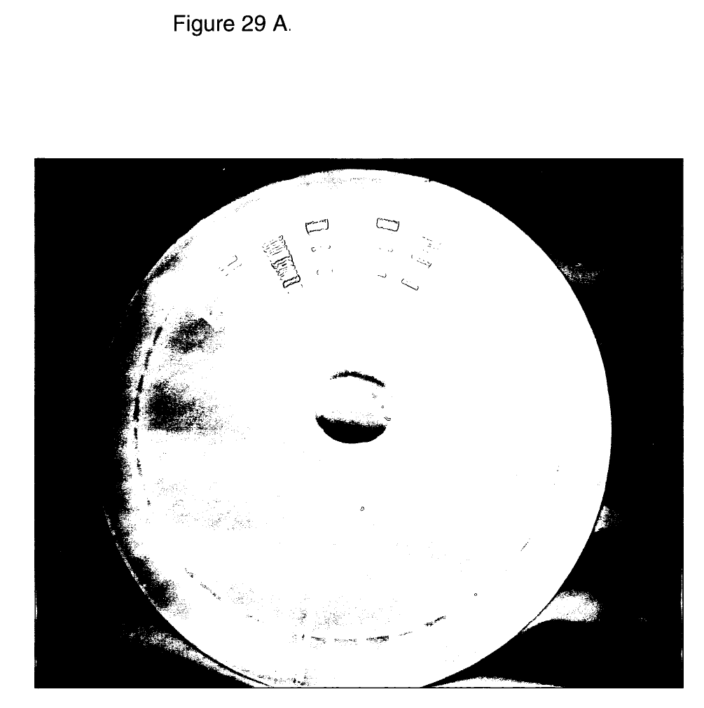

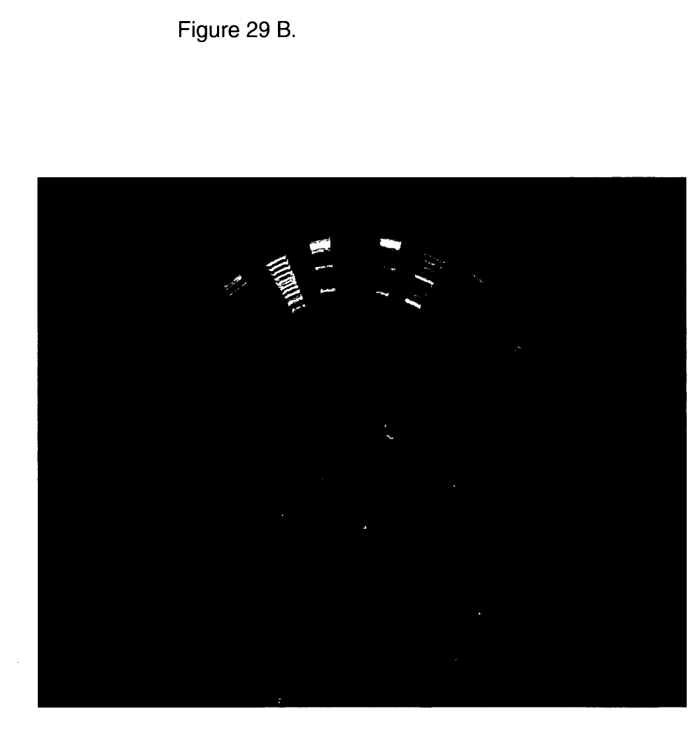

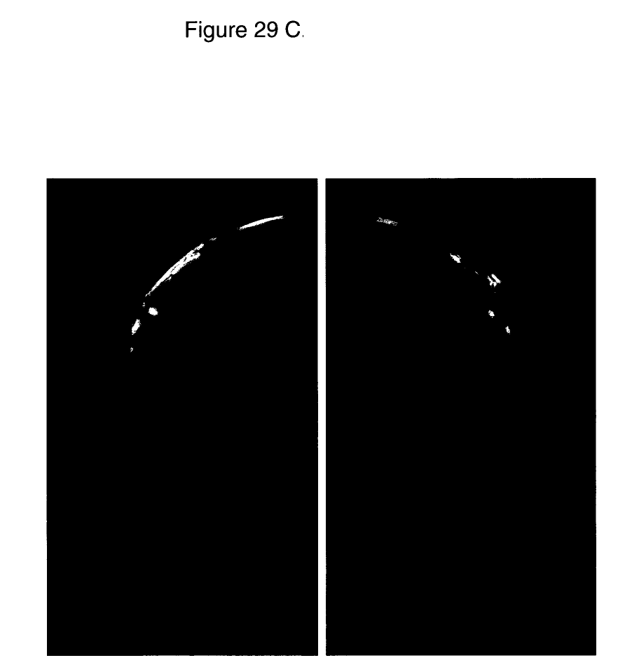

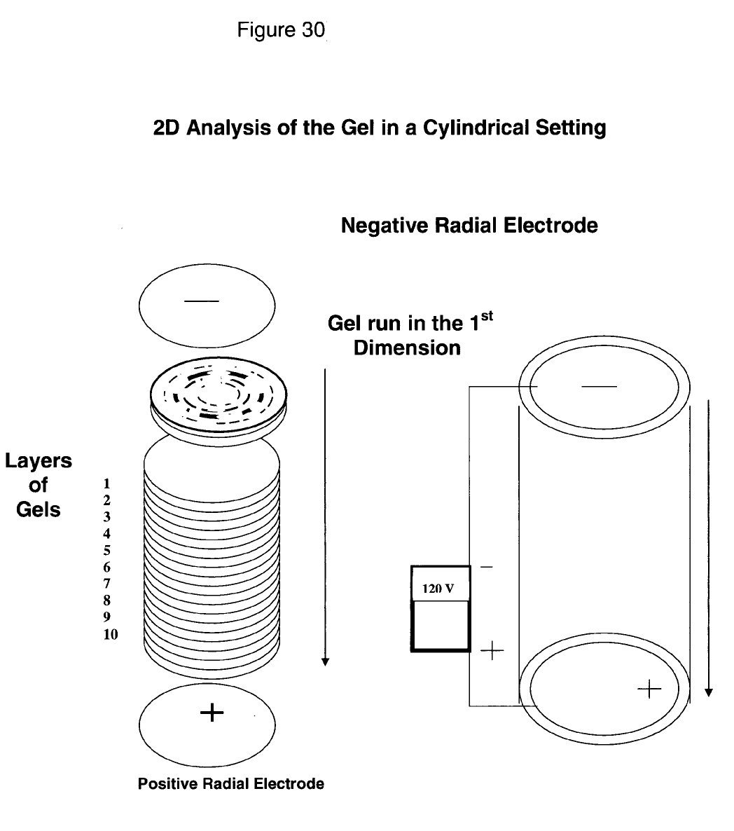

In the other 2-D embodiment of the invention bands are run from radius to radius in a semi circular fashion, where the resolution is higher towards the center. This feature is indicated experimentally (FIG. 27, 28, 29).

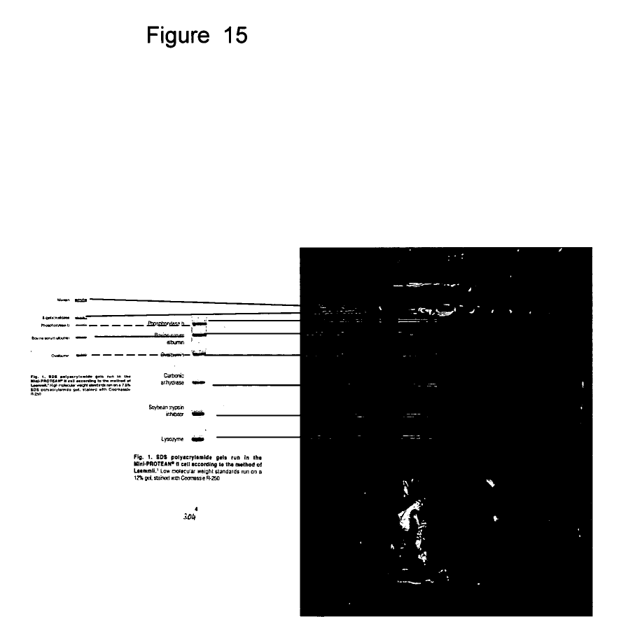

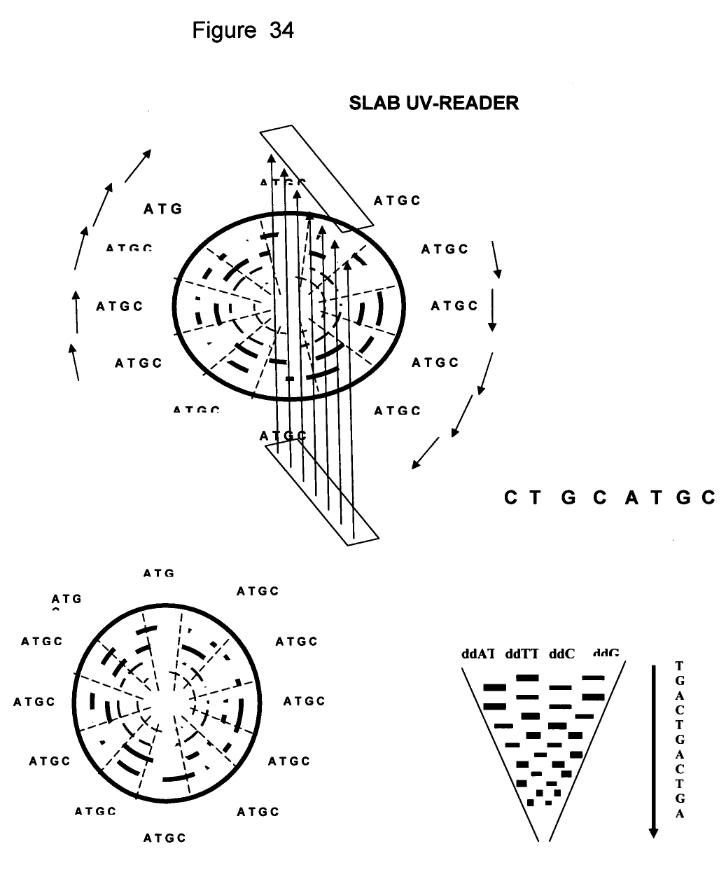

The higher resolution is indicated experimentally on DNA, RNA, and DNA samples on agarose and PAGE backgrounds. The higher resolution for relatively lower molecular weight samples is as indicated (FIGS. 8, 9, 10, 11, 15 and 16). Therefore, the system is a better fit for DNA sequencing (FIG. 34).

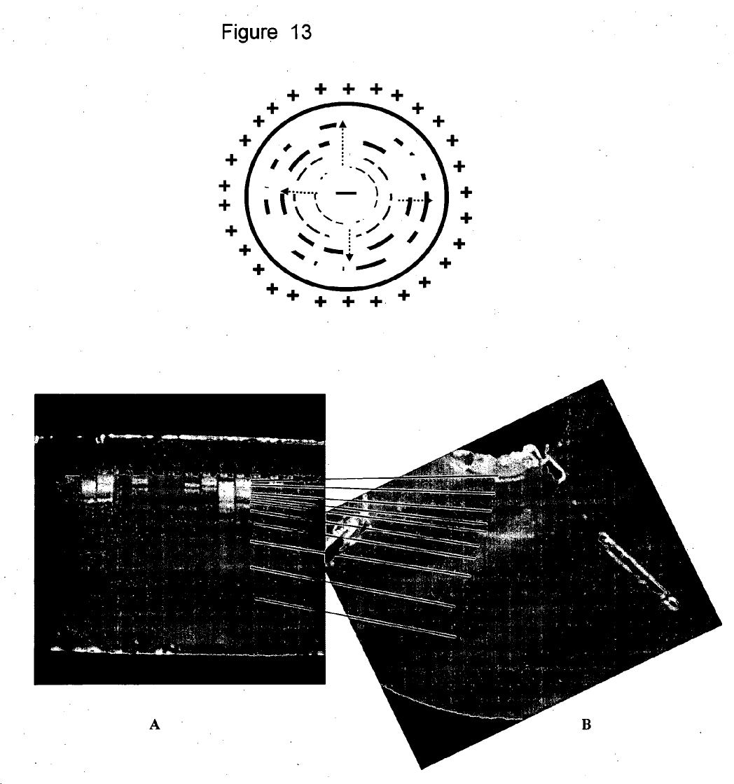

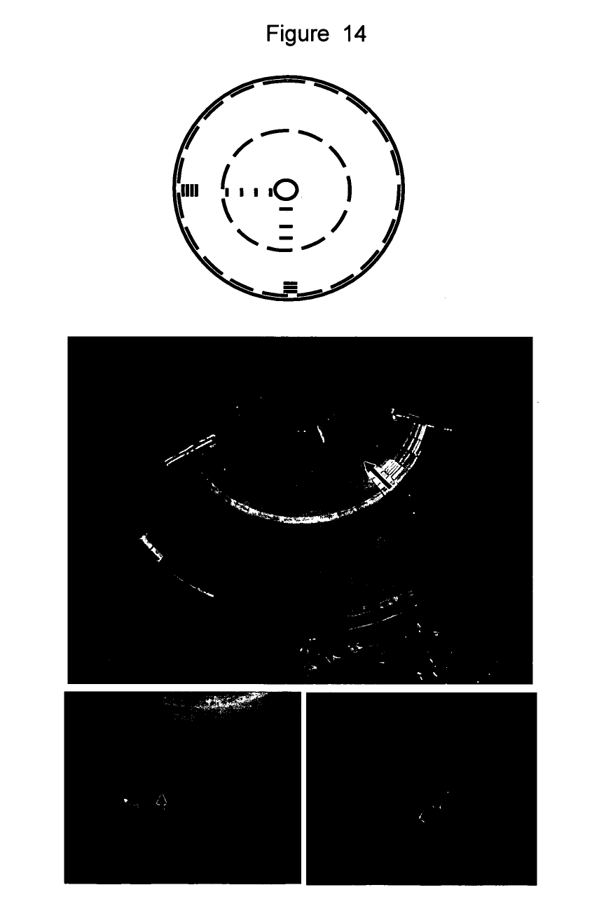

Another aspect of the invention includes higher resolution for high molecular weight samples, when the sample run is in an “inside-out” manner (FIG. 13).

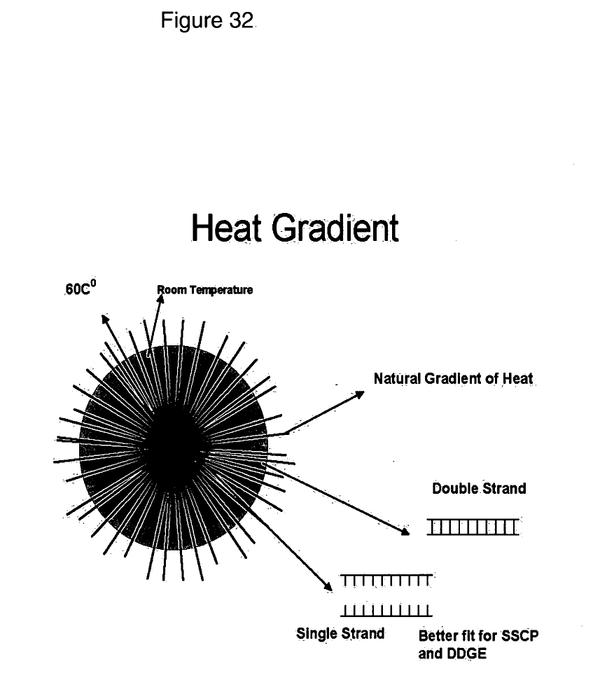

Moderate heating of the central electrode is a convenient feature of the invention for SSCP and DDGE analyses. (FIG. 32).

High band resolution and radial display technique improves sample band resolution to such an extend that gel information as band location (molecular weight), and band intensity can be recorded and read digitally. This is unique to our invention, the radial system.

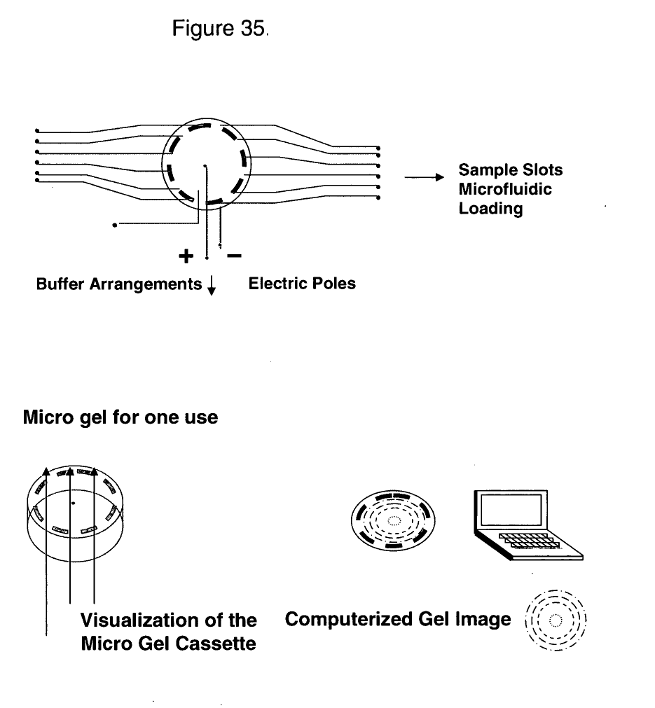

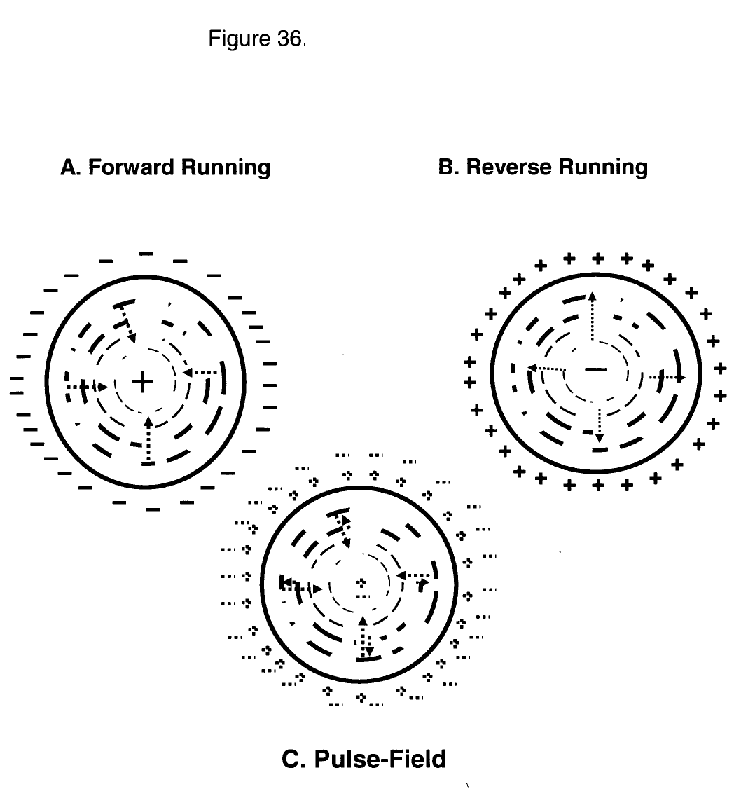

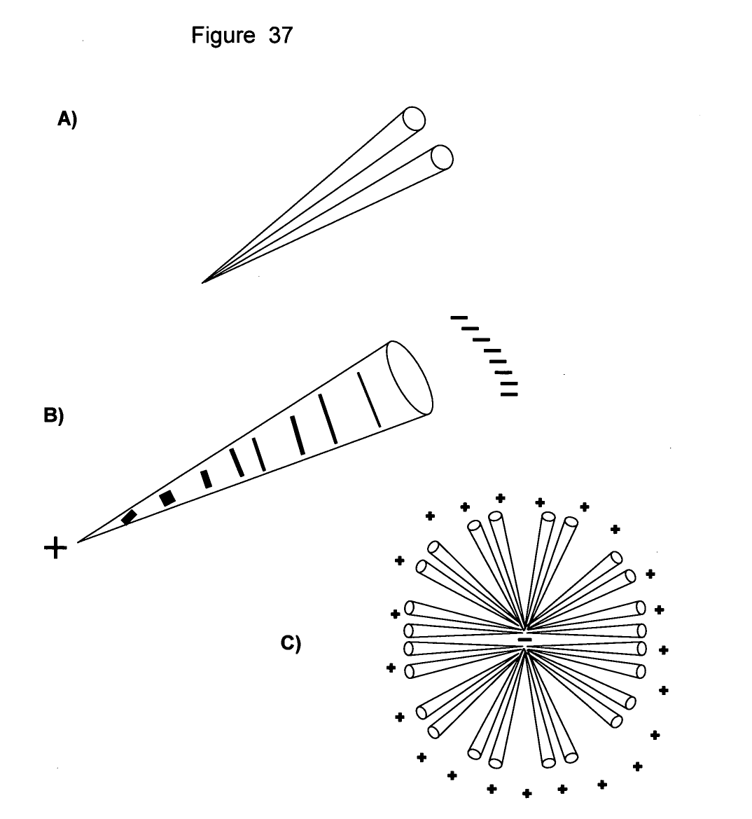

The radial system, including the radial gradient electric field and directional running can be applies to a capillary setting as well as to micro and nano-scales (FIGS. 35, 36, and 37)

All these features facilitate digital processing of the electrophoretic information. When combined with digital recording and reading technology and appropriate software the present invention is promising for the full automation of the electrophoretic systems.

As an analogy, classical rectangular system can be compared to the “A4 paper copy convention”.

1. A method for automated, high resolution, analytical gel electrophoresis, wherein gradient electric field is applied onto molecules of interest along triangular or conical migration paths.

2. An apparatus according of claim 1, wherein the sample migration paths are in triangular or conical form and accessory setup such as combs, casting apparatus, sample loading, sample exit ports and buffer chamber is arrangeable into radial form on triangular/conical sample migration paths.

3. The method according to claim 1, wherein the gel is poured in a radial, conical setting resulting in triangular migration paths.

4. The method according to claim 1, wherein a radial, conical adjustable stacking and casting system is applied, which allows different concentrations and different lengths of gels for the purpose of interest.

5. The method of claim 2, wherein band display of high resolution samples along triangular (conical) migration paths is with radial coordinates.

6. A method, wherein an electric field is a natural electric gradient with an increase in the field strength towards the center along the triangular or conical migration paths.

7. The method of claim 6, wherein other means of gradients such as, heat, pH, are applicable to the radial, conical design in counter or forward combinations to the gradient electric field.

8. The method of claim 7, wherein natural heat gradient due to moderate heating of the central electrode is applied to temperature gradient based mutation screening techniques such as, SSCP and DDGE.

9. The method of claim 5, wherein at the constant well-number, well-length and migration distance the radial display setting with triangular (conical) migration paths requires half the surface area of the conventional linear setting.

10. The method of claim 6, wherein the natural gradient electric field is correlated to decreasing band size and associated decreasing molecular weight, where shorter bands are exposed to a stronger electrical field and resulting electrical force as they approach towards the center along the triangular (conical) migration paths.

11. The method of claim 10, wherein bands representing relatively lower molecular weights, such as primer dimers are narrower in width and more concentrated as they approach to the center along the triangular (conical) migration paths.

12. The method according to claim 1, wherein rotational automation on the gradient electric field background along plurality of triangular (conical) migration paths is used for gel loading, for digital gel visualization and for automated gel extraction.

13. The method according to claim 3, wherein gradient electric field is superimposed onto gel gradient, where gel gradient increases parallel to the gradient electric field.

14. The method according to claim 5, wherein the radial gradient arrangement of triangular (conical) migration paths and the resulting high resolution data display potential forms a basis for further data analysis with advanced in silico techniques such as, machine-learning approaches, including but not limited to neural networks, hidden Markov models, belief networks and support vector machines.

15. The method according to claim 1 wherein the gradient electric field based high resolution electrophoretic system is used to analyse DNA, RNA, polypeptides, polynucleotides, polysaccharides, protein samples, and affinity-purified post-translationally modified (PTM) peptides or protein complexes, as well as to detect a range of reversible or irreversible alterations to amino acid side chains, including but not limited to carbonylation, phosphorylation, glutathionylation, 3-nitrosylation, formation of mixed disulphide, effects on disulphide bridge patterns, ubiquitinylation, esterification, lipoproteins and glycosylation.

16. The method according to claim 1, wherein the gradient electric field based high resolution electrophoretic system is used for the efficient detection of point mutations, transversions, transitions, small deletions and inversions, all of which can be translated into detection of SNPs (Single Nucleotide Polymorphisms) and their combinations.

17. The method according to claim 1, wherein the gradient electric field based high resolution electrophoretic system is used for detection of molecular interactions, such as cofactor-protein (e.g. Fe+-hemoglobin, protein-protein (e.g. sub-domains of T4 lysozyme), protein-DNA (e.g. transcription factor-DNA), protein-RNA (e.g. ribosomal proteins RNA).

18. The method according to claim 5, wherein the high capacity radial system is applied to population studies with large sample groups and statistical analyses, where analysis of high number samples are required on the same platform for statistical purposes.

19. The method according to claim 1, wherein the gradient electric field based high resolution electrophoretic system is used in basic research, including genomics, and proteomics approaches as well as in translational research and in clinical setting.

20. The method according to claim 1, wherein the band intensity and differential molecular weight is correlated to microarray experiments for RNA samples and protein arrays for protein samples.

21. The method according to claim 1, wherein the system is applied to discriminate normal versus disease and normal versus perturbation as an “all or non-event” and as a gradient from normal to perturbation, including the cases “in between”.

22. The method according to claim 1, wherein the system is used at micro and nano-scale.

23. The method according to claim 1, wherein the gradient electric field based high resolution electrophoretic system is used to improve current industrial genomics applications at the following points:
RNA quantization does not always reflect corresponding protein levels.
Multiple proteins can be obtained from each gene.
Genomics can not predict post-translational modifications and the effects thereof.
DNA/RNA analysis cannot predict the amount of a gene product made.
DNA/RNA analysis cannot predict events involving multiple genes.

24. The method according to claim 1, wherein the system is used in diagnostic front of any kind.

25. The method according to claim 1, wherein the radial system is used for the discovery and characterization of bio-markers of the condition of interest.

26. The method according to claim 1, wherein the system is used for 2-D separation.

27. The method of claim 26, wherein the samples are run in the 2nd dimension (2D) in a semicircular-fashion following a gradient electric field in between two poles aligned along the radius, from radius to radius.

28. The method of claim 26, wherein a different 2-dimension (2D) is applied onto biomolecules upon putting the individual gels on top of each other after the first run in a radial setting, where in the second dimension either iso-electric focusing or any-other differential gradient resolution technique is applied to resolve the samples in a cylindrical setting.

29. The method according to claim 1, wherein the application is pertained to non-gradient agarose, polyacryamide (PAGE) (non-equilibrium pH gradient electrophoresis (NEPHGE), including clear native CN-PAGE, and Blue native BN-PAGE, QPNC-PAGE) as well as to gradient polyacrylamide (PAGE) and agarose gels.

30. The method according to claim 1, wherein the gel is visualized with UV, silver staining, coomassie blue staining, by radioactive techniques and by any other possible means.

31. The method according to claim 1, wherein different fluorescent dyes are applied in conjunction with fluorescent interaction systems with multiple fluoro-dyes, such as FRET analysis.

32. The method according to claim 1, wherein reducing conditions with SDS are applied onto the radial gel.

33. The method according to claim 1, wherein the gradient electric filed is correlated to gel composition gradient (gel percentage) in a continuous as well as discontinuous setting.

34. The method according to claim 1, wherein a pulse field, “reverse-forward-reverse-forward” running pattern of the system is applied to the radial gradient electric field.

35. The method according to claim 1, wherein the samples are applied to the center and are exposed to a strong resolution first time they encounter the electric field and the gel.

36. The method according to claim 35, wherein the “inside-out” running is used for high resolution dissection of biomolecules with high molecular weight, such as, chromosomal segments, (E.g. 21st chromosome in down syndrome), and on any other high molecular differential setting.

37. The method according to claim 1, wherein RNA, DNA, or protein samples are transferred to the radial blotting medium of interest.

38. The method according to claim 1, wherein the system is used for isoelectric focusing (IEF) and for any other differential separation technique.

39. The method according to claim 1, wherein the isolated RNA, DNA, protein, polysaccharide, polypeptide samples are used in conjunction with further analyses for identification and characterization purposes including but not limited to mass-spectrophotometry.

40. The method according to claim 5, wherein automated sample loading making use of radial structure is applicable onto the system along triangular migration paths, where automated radial-rotation of the gel by “one well-degree” facilitates robotic arm functioning and automated loading.

41. The method of claim 1, wherein the system is visualized by automated UV and illumination exposure and by associated electronic scanning and recording of the generated picture.

42. The method of claim 40, wherein the images are correlated with an automated excision system moving in a rotational as well circumferential-central setting.

43. The method according to claim 5, wherein comparative samples (E.g., normal versus disease) are loaded on 180 degree opposite sites, symmetrical to a common axis.

44. The method according to claim 5, wherein the gel or the gel image is rotated and the resulting band strengths is translated into differential high sample resolution in a bar-code setting along triangular migration paths.

45. The method according to claim 1, wherein automated cross database comparison is applied onto the system, where a universal single dimension factor −R and molecular weight standards are used to compare the images universally from different labs with the radius (R) taken as the only, single dimension as a reference for data correlation across different gel sizes.

46. The method according to claim 5, wherein originated images are “cut out insilico” and compared to each other in a symmetrical setting.

47. The method according to claim 1, wherein radial electric field is used in capillary electrophoresis with capillaries formulated in a conical form resulting in a gradient electric field in conical capillaries.

48. The method according to claim 1, wherein gradient electric filed concept and associated high resolution electrophoretic potential is applied to conical separation capillaries with broader loading-ends and narrower exit-ends and vice versa.

49. The method according to claim 1, wherein a reverse running-system is used to select for DNA, RNA and protein samples larger than the minimum size.

50. The method of claim 5, wherein bands falling off the gel at the center are collected at the central indentation for analytical purposes including but not limited to mass spectrophotometry.

51. The method according to claim 1, wherein the approach is applied to differential profiling of DNA markers such as VNTR, microsattelite, and RFLP analysis.

52. The method according to claim 1, wherein continuous running of the system is correlated with a continuous illumination-reader for simultaneous, real-time band localization.
